(19) United States
(12) Patent Application Publication
(10) Pub. No.: US 2009/0170930 A1
(43) Pub. Date: Jul. 2, 2009

(54) METHODS FOR DIRECTING DIFFERENTIATION OF CLONOGENIC NEURAL STEM CELLS WITH COUMARINS

(75) Inventors: Cheng HE, Shanghai (CN); Weidong ZHANG, Shanghai (CN); Xiaohui XU, Shanghai (CN); Wei ZHANG, Shanghai (CN); Juan SU, Shanghai (CN); Chuan ZHANG, Shanghai (CN)

Correspondence Address: MATTHIAS SCHOLL, 14781 MEMORIAL DRIVE, SUITE 1319, HOUSTON, TX, 77079, US

(73) Assignee: THE SECOND MILITARY MEDICAL UNIVERSITY, Shanghai (CN)

(21) Appl. No.: 12/400,756

(22) Filed: Mar. 9, 2009

Related U.S. Application Data
(63) Continuation of application No. PCT/CN2007/002638, filed on Sep. 3, 2007.

(30) Foreign Application Priority Data
Sep. 7, 2006 (CN) 200610030917.1

(51) Int. Cl. A61K 31/35

(52) U.S. Cl. 514/457

(57) ABSTRACT

A method for promoting differentiation of clonogenic neural stem cells (NSCs), comprising administering to a patient in the need of such promoting a coumarin compound represented by formula I or by formula II. The representative coumarin compounds include 7-hydroxycoumarin, daphnoretin, scopoletin, edgeworin, aesculetin and esculetin-6-β-D-glucopyranoside. The coumarin compounds showed significant activity of directing the differentiation of NSCs in pharmacological test and thereof could be used to prepare drugs to direct NSCs differentiated to oligodendrocyte progenitor cells (OPCs) for the treatment of demyelinating diseases or spinal cord injury. The drug could be a pure coumarin compound or a pharmaceutical composition comprising a therapeutical dose of a coumarin compound as active ingredients and a pharmaceutically-acceptable carrier. The content of the active ingredients in the pharmaceutical composition is between 0.1% and 99.5% by weight.

### CROSS-REFERENCE TO RELATED APPLICATIONS

This application is a continuation of International Patent Application No. PCT/CN2007/002638 with an international filing date of Sep. 3, 2007, designating the United States, now pending, and further claims priority benefits to Chinese Patent Application No. 200610030917.1, filed on Sep. 7, 2007. The contents of these specifications are incorporated herein by reference.

### BACKGROUND OF THE INVENTION

1. Field of the Invention

The present invention relates to methods for directing the differentiation of clonogenic neural stem cells (NSCs) with coumarins.

2. Description of the Related Art

Clonogenic neural stem cells (NSCs) are self-renewing cells that maintain the capacity to differentiate into brain specific cell types, such as neurons, astrocytes, and oligodendrocytes, and may also replace or repair diseased brain tissue. The neural stem cells are abounding in the fetal or adult spinal cord and in the third and fourth ventricles. These cells persist in the subventricular zone, hippocampus, cortex, and spinal cord, even in the adult.

Neural stem cells (NSCs) are immature cells with the ability to renew themselves and give rise to neurons, astrocytes, and oligodendrocytes. Isolated NSCs are able to proliferate in response to basic fibroblast growth factor or epidermal growth factor, and when the culture conditions are altered, they differentiate into several phenotypes of neurons. Furthermore, neurons derived from NSCs form functional synapses in vitro and in vivo. These results suggest that NSCs have the potential to differentiate into appropriate neurons to form a functional neuronal circuitry.

Transplants of human brain-derived stem cells or human spinal cord tissue into injured rat spinal cord have been described (Wictorin, K. & Bjorklund, A. (1992) NeuroReport 3, 1045-1048. Stepanov, G. A., Karpenko, D. O., Aleksandrova, M. A., Podgornyi, O. V., Poltavtseva, R. A., Pevishchin, A. V., Marey, M. V. & Sukhikh, G. T. (2003) Bull. Exp. Biol. Med. 135, 397-400. Akesson, E., Holmberg, L., Jonhagen, M. E., Kjaeldgaard, A., Falci, S., Sundstrom, E. & Seiger, A. (2001) Exp. Neurol. 170, 305-316.).

Moreover, several human cell transplantation paradigms recently have been reported to promote locomotor recovery: human umbilical cell infusion in a rat spinal cord injury model, although only within 3 weeks or less postgrafting (Saporta, S., Kim, J. J., Willing, A. E., Fu, E. S., Davis, C. D. & Sanberg, P. R. (2003) J. Hematother. Stem Cell Res. 12, 271-278.); neurons differentiated in vitro under retinoic acid from human embryonal teratocarcinoma cells and transplanted into a rat spinal cord injury model (Saporta, S., Makoui, A. S., Willing, A. E., Daadi, M., Cahill, D. W. & Sanberg, P. R. (2002) J. Neurosurg. 97, 63-68.); human ES cells differentiated in vitro to oligoprogenitors and transplanted into a rat spinal cord injury model (Keirstead, H. S., Nistor, G., Bemal, G., Totoiu, M., Cloutier, F., Sharp, K. & Steward, O. (2005) J. Neurosci. 25, 4694-4705.); and human neural stem progenitor cells transplanted into a monkey spinal cord injury model (Iwanami, A., Kaneko, S., Nakamura, M., Kanemura, Y., Mori, H., Kobayashi, S., Yamasaki, M., Momoshima, S., Ishii, H., Ando, K., et al. (2005) J. Neurosci. Res. 80, 182-190.)

However, spontaneous differentiation of neural stem cells (NSCs) is generally inefficient and mainly leads to large amount of astrocytes, which are not useful for cell-based therapy (Cao et al. 2001).

Demyelination is a prominent feature of many CNS disorders, including multiple sclerosis (MS), spinal cord injury (SCI) and stroke (Mason et al. 2004, Tanaka et al. 2003, Keirstead et al. 2005). The currently available treatments aim at preventing future demyelination based on immunomodulation or immunosuppression (Bernd et al. 2005), and have so far been only partly successful by reducing disease progression without stopping further demyelination (Chari and Blakemore 2002). Since the primary defect appears to be demyelination, the most straightforward approach is to provide new myelinating cells for compensation within the lesions (Groves et al. 1993). Given the greater mitotic and migratory potential of oligodendrocyte progenitor cells (OPCs), OPCs are the preferred cell for myelin repair (Chari and Blakemore 2002).

Although human OPCs have been isolated from both abortion-derived and adult human CNS (Aloisi, et al. 1992, Armstrong, et al. 1992), it is obviously difficult to acquire sufficient donor cells and ethically unlikely ever to be acceptable. OPC cell lines as an unlimited cell source have been generated and shown to remyelinate experimental lesions (Barnett, et al. 1993), but uncontrolled cell proliferation of a cell line could be a limiting factor for a therapeutic strategy based on cell transplantation.

Clearly, more efficient and selective approaches are needed to direct the differentiation of NSCs, to produce homogenous populations of OPCs.

### SUMMARY OF THE INVENTION

In view of the above-described problems, it is one objective of the invention to provide methods for directing the differentiation of clonogenic neural stem cells (NSCs).

It is another objective of the invention to provide a method for treating demyelinating diseases or spinal cord injury.

It is another objective of the invention to provide a pharmaceutical composition comprising a carrier and a coumarin compound of the formula I or II as active ingredients.

The formula I is shown below,

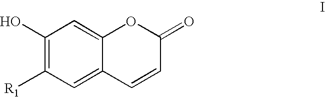

wherein R₁ represents hydrogen, hydroxyl, phenyl, or a glycosidic moiety comprising 1-5 sugars.

The formula II is shown below,

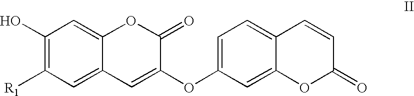

wherein R₁ represents hydrogen, hydroxyl, phenyl, or a glycosidic moiety comprising 1-5 sugars.

The coumarins disclosed in the invention are distributed in many plant species. These coumarins could be obtained by extraction from plants or by chemical synthesis.

The representative compounds of the coumarins disclosed in this invention include, without limitation, 7-hydroxycoumarin, daphnoretin, scopoletin, edgeworin, aesculetin, and esculetin-6-β-D-glucopyranoside.

Previously, we purified and identified more than 2,000 natural compounds from Traditional Chinese Medicine (TCM) and built up a natural products library. The high-throughput screening of the library lead to the discovery of the coumarins disclosed in the invention which exhibit the activity of directing differentiation of clonogenic neural stem cells (NSCs). Further studies demonstrated that these coumarin compounds could promote NSCs differentiation to oligodendrocyte precursor cells (OPCs).

In certain embodiments of the present invention, the pharmaceutical composition disclosed herein comprises a therapeutical dose of a coumarin compound as active ingredient and a pharmaceutically acceptable carrier. The content of the active ingredient is between 0.1% and 99.5% by weight.

In certain embodiments of the present invention, the compounds and the pharmaceutical compositions disclosed herein are administered to a patient to direct NSCs differentiation to OPCs whereby treating a demyelinating disease or spinal cord injury.

In certain embodiments of the present invention, the pharmaceutically acceptable carrier comprises a conventional drug carrier, such as a diluent (water, starch, sugar, etc.), an adhesive (cellulose derivatives, alginate, gelatin, polyvidone, etc.), a wetting agent (glycerol, etc.), a disintegrant (agar, CaCO3, NaHCO3, etc.), a penetration enhancer (quaternary ammonium compound, etc.), a surfactant (palmityl alcohol, etc.), an adsorbent (kaolin, bentonite, etc.), and/or a lubricant (talc, calcium stearate, magnesium stearate, macrogol, etc.). Other excipients, such as scenting agents and sweetening agents can also be added to the pharmaceutical composition.

In certain embodiments of the present invention, the compounds and pharmaceutical compositions disclosed herein are administered to a patient in need of such treatment orally, intranasally, rectally, or parenterally. When given orally, the compounds or pharmaceutical compositions can be prepared in tablets, powder, granule, gelatin capsule, or liquid preparations (water suspension, oil suspension, syrup, elixir, etc.). When given parenterally, the compounds or pharmaceutical compositions can be prepared in solution, water suspension, or oil suspension for injection. The preferred form is tablet, coated tablet, gelatin capsule, suppository, nasal spray, or injection.

In certain embodiments of the invention, the dosage forms of the compounds and pharmaceutical compositions disclosed herein can be prepared by conventional methods used in the pharmaceutical industry.

### BRIEF DESCRIPTION OF THE DRAWINGS

Detailed description will be given below with reference to accompanying drawings, in which:

FIG. 1 is a cell morphogram of NSCs treated with 7-hydroxycoumarin;

FIG. 2 is a cell morphogram of NSCs treated with daphnoretin; and

FIG. 3 is a cell morphogram of NSCs treated with DMSO.

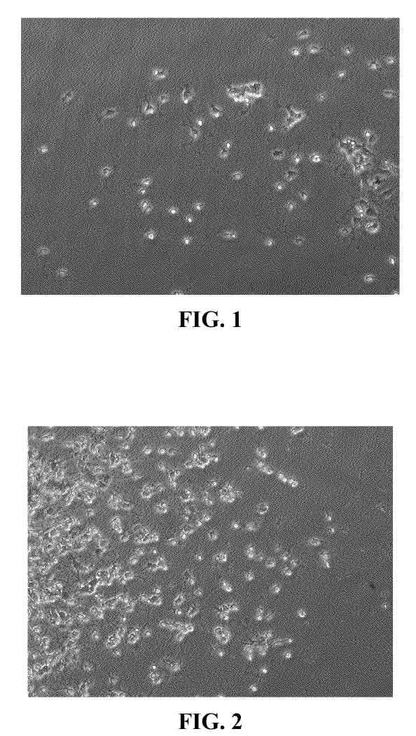

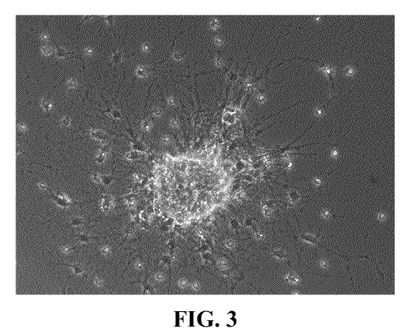

### DETAILED DESCRIPTION OF THE EMBODIMENTS

Examples of preparations and results of pharmacological test according to the present invention are shown below. The Examples do not limit the scope of the invention, but are intended to make the invention more clearly understandable.

Preparation of Compounds:

### Example 1

### 7-hydroxycoumarin

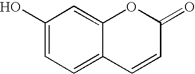

Roots of Daphne giraldii Nitsche (11 kg) were air-dried and chopped into small pieces. Ethanol extract of the roots (95% v/v) was prepared and evaporated in vacuo. The residue was suspended in water, and then partitioned with petroleum ether, chloroform (CHCl₃), ethyl-acetate (EtOAc) and n-butyl alcohol (n-BuOH). The four fractions were concentrated in vacuo and stored at −20° C. prior to further purification. The EtOAc fraction was subjected to silica gel chromatography. The eluents for this fraction on column chromatography on silica gel were different ratios of CHCl₃ and MeOH, as mobile phase to afford nine sub-fractions. Sub-fraction 2 was further purified by silica gel chromatography to afford a single compound (white powder). The compound was identified as 7-hydroxycoumarin using UV, IR, mass, and NMR spectra.

### Example 2

### Daphnoretin

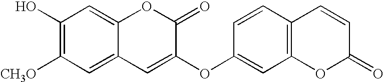

Roots of Daphne giraldii Nitsche (11 kg) were air-dried and chopped into small pieces. The ethanol extract of the roots (95%, v/v) was prepared and evaporated in vacuo. The residue was suspended in water, and then partitioned with petroleum ether, chloroform (CHCl₃), ethyl-acetate (EtOAc) and n-butyl alcohol (n-BuOH). The four fractions were concentrated in vacuo and stored at −20° C. prior to further purification. The EtOAc fraction was subjected to silica gel chromatography. The eluants for this fraction on column chromatography over silica gel were different ratios of CHCl₃ and MeOH, as mobile phase to afford nine sub-fractions. Sub-fraction 5 was further purified on silica gel chromatography to afford a single compound (yellow powder). The compound was identified as daphnoretin using UV, IR, mass, and NMR spectra.

### Example 3

### Scopoletin

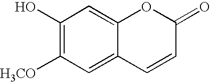

Roots of Daphne odora Thunb. var. atrocaulis Rehd. (5 kg) were air-dried and chopped into small pieces. The roots were then percolated with ethanol (75%, v/v). The ethanol extract was evaporated in vacuo. The residue was suspended in water, and then partitioned with petroleum ether, chloroform (CHCl₃), ethyl-acetate (EtOAc) and n-butyl alcohol (n-BuOH). The four fractions were concentrated in vacuo and stored at −20° C. prior to further purification. The CHCl₃ fraction was subjected to column chromatography on silica gel, Sephadex LH-20 and HPLC to afford 12 compounds. One of these compounds (yellow powder) was identified as scopoletin using UV, IR, mass, and NMR spectra.

### Example 4

### Edgeworin

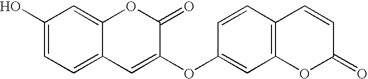

Roots of Edgeworthia chrysantha (5 kg) were air-dried and chopped into small pieces. Ethanol extract (80% v/v) was prepared and evaporated in vacuo. The residue was suspended in water, and then partitioned with petroleum ether, chloroform (CHCl₃), ethyl-acetate (EtOAc) and n-butyl alcohol (n-BuOH). The four fractions were concentrated in vacuo and stored at −20° C. prior to further purification. The CHCl₃ fraction was subjected to column chromatography on silica gel, Sephadex LH-20 and HPLC to afford 12 compounds. One of the compounds (yellow powder) was identified as edgeworin using UV, IR, mass, and NMR spectra.

### Example 5

### Aesculetin

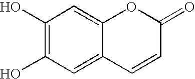

The air-dried and powdered stem bark of Fraxinus rhynchophylla Hance. (15 kg) was extracted 3 times with 40 L ethanol each time for 2 h. The solvent was evaporated in vacuo. The residue was suspended in water, and then partitioned with petroleum ether, chloroform (CHCl₃), ethyl-acetate (EtOAc) and n-butyl alcohol (n-BuOH). The four fractions were concentrated in vacuo and stored at −20° C. The EtOAc extract was subjected to column chromatography on silica gel (200-300 mesh) and eluted successively with gradient CHCl₃-MeOH mixtures of increasing polarity to give five compounds. One of the compounds was identified as aesculetin using UV, IR, mass, and NMR spectra.

### Example 6

### Esculetin-6-β-D-glucopyranoside

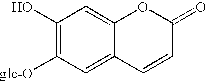

The air-dried and powdered stem bark of Fraxinus rhynchophylla Hance. (15 kg) was extracted three times with 40 L of ethanol, each time for 2 h. The solvent was evaporated under vacuum. The residue was suspended in water, and then partitioned with petroleum ether, chloroform (CHCl₃), ethyl-acetate (EtOAc) and n-butyl alcohol (n-BuOH). The four fractions were concentrated in vacuo and stored at −20° C. The EtOAc extract was subjected to column chromatography on silica gel (200-300 mesh) and eluted successively with gradient CHCl₃-MeOH mixtures of increasing polarity to give five compounds. One of the compounds was identified as esculetin-6-β-D-glucopyranoside using UV, IR, mass and NMR spectra.

### Example 7

### Preparation of Dosage Forms

Tablets:
Active ingredient (a compound listed in Table 1): 20 mg
Lactose: 177 mg
Amylum maydis: 50 mg
Magnesium stearate: 3 mg

Preparation:

The active ingredient is mixed with lactose and amylum maydis. The mixture is wetted by water uniformly. The wetted mixture is filtered, dried and filtered again. Magnesium stearate is added in the mixture before tabletting. There is 20 mg active ingredient in each tablet of 250 mg.

### Example 8

### Pharmacological Testing

Coumarins Promotes Differentiation of NSCs

NSCs Culture

All animal care procedures follow recommended NIH guidelines also approved by the Animal Experimentation Ethics Committee of Second Military Medical University.

Rat NSCs isolation and culture were performed as described by Weiss et al (Weiss et al., 1996). Briefly, the cortex of 1- to 2-day-old newborn SD rats were removed and placed into sterile chilled Hank's Balanced Salt Solution. After the meninges were carefully removed, tissues were cut into tiny pieces and gently triturated with a Pasteur pipette at least 10-15 times. The suspended tissue was centrifuged at 500 g for 5 min and the pellets were re-suspended in a proliferation medium (DMEM/F12 nutrient (1:1) with additional 2% B27 (Invitrogen Corporation, Carlsbad, Calif.) supplement and 20 ng/mL bFGF (PeproTech, Rocky Hill, N.J.)). The cells were plated at a density of 50 cells/mL in non-coated 30 cm flasks. Every 4 days, bFGF was added along with a partial medium change. After 7 days in vitro, cells formed floating neurospheres. To subculture NSCs, the neurospheres were centrifuged at 600 g for 5 min when the diameters of neurospheres reached a size of approximately 100-200 μm; they were then re-suspended in fresh medium and mechanically dissociated into single cells. Single cells subsequently were seeded into proliferation medium at a density of 10 cells/mL per flask. This procedure produced a second generation of neurospheres, and additional generations of NSCs were produced using this same procedure.

Compound Screens.

Neurospheres were plated into 12-well plates on poly-D-lysine coated 35 mm dishes at a density of 10-20 spheres per dish in 1.5 mL medium. 1 hour after plating, compounds were added at a final concentration 100 μM unless otherwise indicated. After 5 days, cells were examined with Phase contrast image or immunocytochemistry.

Immunocytochemistry and Cell Count Assessment

Identification of the different kinds of cells was performed using indirect immunocytochemistry. Cells on dishes were fixed with 4% paraformaldehyde at room temperature for 20 min and washed three times in succession with 0.01 M PBS for 5 min each time. Cells were then treated with 0.3% Triton 100 containing 10% normal goat serum at room temperature for 30 min (Xu et al., 2006). Cells were then incubated with primary antibodies at 4° C. for 12-16 h and washed three times with 0.01 M phosphate buffered saline (PBS) for 5 min each time. FITC-conjugated secondary antibodies were added to the cells, and the cells were incubated at 37° C. for 40 min. After three 5 min PBS washes, Hochest 33258 nuclear stain was added at room temperature for 10 min, followed by two more 5 min PBS rinses. The dishes were examined by a fluorescence microscopy. The primary antibodies included anti-GFAP (1:200; rabbit; Promega), anti-nestin (1:500; Mouse; Chemicon); anti-NG2(1:200; Mouse; Chemicon), anti-MBP (1:50; Mouse; Chemicon), anti-04 (1:100; Mouse; Chemicon), anti-RIP (1:100; Mouse; Chemicon), anti-olig2 (1:100; rabbit; Abcam). The secondary antibodies were FITC-labeled antibodies to rabbit IgG (1:200) and TRITIC-labeled antibodies to mouse IgG (1:200). Immunostained preparations were examined with an IX70 Olympus microscope equipped for phase contrast and epi-fluorescence. Light from a 75 W xenon arc lamp (AH2-RX, Olympus) passed through filter sets (Chroma) to generate excited light for Hochest 33258, FITC and TRITIC stains. All image acquisitions were finished with computer-controlled CCD (Micromax 5 MHz system, Roper Princeton Instruments) and MetaMorph Imaging System version 3.6 (Universal Imaging Corporation). To determine the number of cells expressing a particular antigen, 100 fields per sample were examined and totaled. Observers without knowledge of the treatment condition counted the numbers of positive cells for NG2, compared to total numbers of progeny of NSCs determined by counting Hochest 33258 staining cell nuclei. Results are given as mean±S.D. of data for six samples from three independent experiments. Statistical analyses were carried out using Student's t-test with SPSS software (Version 10.1).

Results

Daphnoretin Promotes Generation of NG2 Positive Cells from NSCs.

NSCs are generally considered as tri-potent, self-renewing progenitors that can generate neurons, astrocytes and oligodendrocytes, the three major cell types of the CNS (Weiss et al., 1996). To identify small molecules that can induce the selective differentiation of NSCs, we carried out a screen based on a compound library purified from traditional Chinese herbs. To carry out the primary screen, NSCs were treated with 100 μM (final concentration) compounds after plating in neurobasal with 2% B27. After 5 days, cultures were examined under phase contrast microscope.

We found that daphnoretin can markedly enhance the generation of round bipolar or multipolar morphology cells (data not shown), probably OPCs. To further confirm the effect of daphnoretin, Nestin positive second-generation neurospheres was plated on cell culture dish and cultured for 10 days with 100 μM daphnoretin. Cells that migrated out of the neurosphere and displayed a bipolar or multipolar morphology characteristic of OPCs, and this characteristic will keep for another 10 days (data not show). We next performed immunostaining for these cells with NG2 (a specific marker for OPCs) and GFAP (a specific marker for astrocytes), and the results showed that daphnoretin treatment dramatically increased the generation of NG2 positive cells. NG2 positive cells were 21.91% of the total cells treated with DMSO, whereas NG2 positive cells were 65±5.3%, 87±7.5%, 92±8.6%, 95±6.9% when treated with daphnoretin in 1 μM, 10 μM, 100 μM, and 1 mM, respectively.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

TABLE 1
The ratio of NG2 positive cells of the total cells (%)

| | | | Dose | | | |
|---|---|---|---|---|---|---|
| No. | Name | Structure | 1 mM | 100 μM | 10 μM | 1 μM |
| 1 | DMSO | | 21 ± 2.5 | 20 ± 3.1 | 23 ± 4.2 | 24 ± 2.6 |
| 2 | Genkwanin | 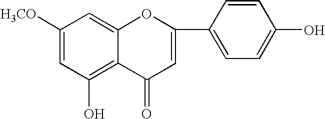 | 1 | 1 | 1 | 23 ± 2.3 |
| 3 | Aesculetin | 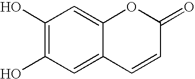 | 75 ± 1.6* | 68 ± 2.7* | 63 ± 2.9* | 53 ± 2.4* |
| 4 | 7-hydroxycoumarin | 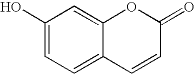 | 92 ± 8.7* | 89 ± 9.3* | 76 ± 6.6* | 68 ± 6.5* |
| 5 | Daphnoretin | 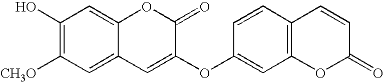 | 95 ± 6.9* | 92 ± 8.6* | 87 ± 7.5* | 65 ± 5.3* |
| 6 | Edgeworin | 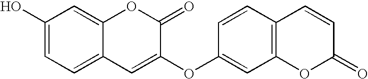 | 81 ± 3.1* | 68 ± 3.5* | 63 ± 2.9* | 60 ± 2.6* |
| 7 | Esculetin-6-β-D- glucopyranoside | 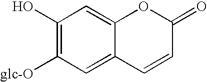 | 85 ± 5.2* | 70 ± 3.9* | 72 ± 3.5* | 67 ± 4.6* |
| 8 | Scopoletin | 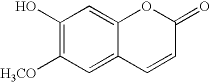 | 68 ± 6.7* | 70 ± 6.9* | 63 ± 6.3* | 62 ± 5.6* |
| 9 | Isopsoralen | 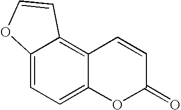 | 1 | 1 | 1 | 21 ± 1.6 |
| 10 | Aesculin | 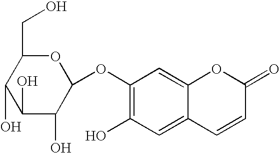 | 1 | 1 | 1 | 19.8 ± 2.3 |
| 11 | 7-methoxy coumarin | 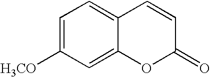 | 1 | 1 | 1 | 16.7 ± 2.2 |
| 12 | Isodaphnoretin | 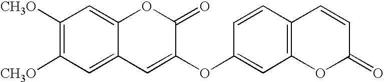 | 1 | 1 | 1 | 15.6 ± 1.8 |
| 13 | Daphnoretin-7-O-β- D-glucopyranoside | 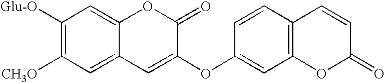 | 1 | 1 | 1 | 1 |
| 14 | Edgeworoside C | 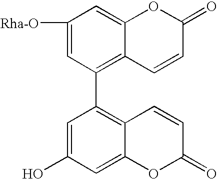 | 1 | 1 | 1 | 1 |
| 15 | Dalbergin | 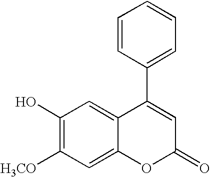 | 1 | 1 | 1 | 1 |

*compared with DMSO, P < 0.5.

The invention claimed is:

1. A method for promoting differentiation of clonogenic neural stem cells (NSCs), comprising administering to a patient in the need of such promoting a coumarin compound represented by formula I or by formula II, 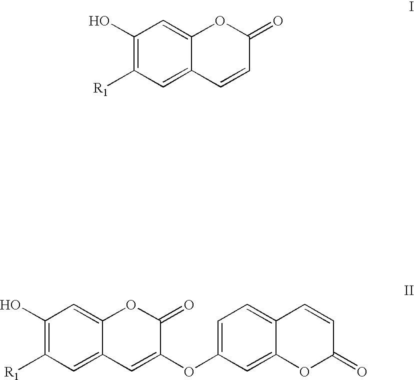
wherein
R₁ at each occurrence represents hydrogen, hydroxyl, phenyl, or a glycosidic moiety comprising 1-5 sugars.

2. A method for promoting differentiation of clonogenic neural stem cells (NSCs), comprising administering to a patient in the need of such promoting a pharmaceutical composition comprising an excipient and a coumarin compound represented by formula I or by formula II, 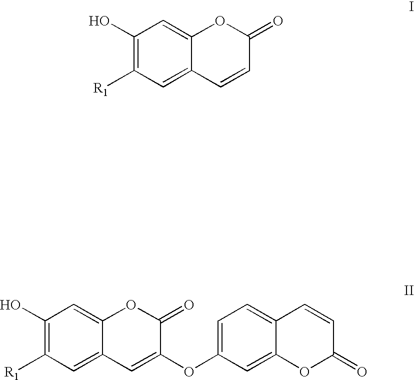
wherein
R₁ at each occurrence represents hydrogen, hydroxyl, phenyl, or a glycosidic moiety comprising 1-5 sugars.

3. The method of claim 2, wherein said pharmaceutical composition comprises between 0.1% and 99.5% of said coumarin compound by weight.

4. The method of claim 1, whereby by promoting differentiation of clonogenic neural stem cells (NSCs) treating a demyelinating disease or a spinal cord injury.

5. The method of claim 2, whereby by promoting differentiation of clonogenic neural stem cells (NSCs) treating a demyelinating disease or a spinal cord injury.

6. The method of claim 1, wherein said coumarin compound administered to a patient has been prepared by extraction from a plant or by chemical synthesis
